(19) United States
(12) Patent Application Publication
(10) Pub. No.: US 2005/0163687 A1
(43) Pub. Date: Jul. 28, 2005

(54) Device for the sterilization of hospital waste

(75) Inventor: Massimo Del Bo, Rovigo (IT)

Correspondence Address: Serafini Associates, 7660 FAY AVE. STE H378, LA JOLLA, CA, 92037, US

(73) Assignee: ECONOS S.r.l., Rovigo (IT)

(21) Appl. No.: 10/503,514

(22) PCT Filed: May 9, 2002
(86) PCT No.: PCT/IT02/00311

(30) Foreign Application Priority Data
Feb. 5, 2002 (IT) PD2002A000027

(51) Int. Cl. A61L 2/12

(52) U.S. Cl. 422/309; 422/21; 219/679

(57) ABSTRACT

A system for the sterilization of hospital waste, comprising a triturator that includes a hermetic cover, a hopper and toothed drums; an auger feeder to transport the triturated waste and comprising nozzles that blow steam into the waste; a high-temperature pressurized steam generator; an airtight sterilization chamber with microwave generators and with an inner stirrer, the sterilization chamber comprising a gate at the top, a gate on one side or in the front, and a stirring device for mixing the contents of the chamber. During the whole process the waste is completely insulated from the external environment. A central unit controls and supervises the operation of all the electric, electronic and mechanical parts of the system, which can be fit into a modular container for easy transportation.

### BACKGROUND OF THE INVENTION

1. Field of the Invention

The present invention relates to a device for the treatment and disposal of special waste. More particularly, the present invention relates to-new device for the sterilization of potentially infected hospital waste.

2. Description of Related Art

Hospital waste cannot be directly discharged in dumps, like common solid urban waste, because it is highly infectious.

At present, hospital waste must be incinerated in special treatment systems and health organizations have to bear all the direct and indirect expenses connected to these treatments.

In any case, the risk of infection is always present during transportation to the incinerator and other material handling carried out by the various personnel, unless suitable and particularly expensive protective measures are adopted.

Hospital waste is generally closed in special containers in order to protect the personnel that must handle it, and comprises heterogeneous materials, like gauze and tampons, and closed elements, like syringes and bottles, that prevent sterilizing substances from having the desired effect on each single part of the waste.

The known sterilization equipment and devices, like for example UV irradiating equipment, do not ensure the necessary sterilization of hospital waste, since this type of equipment was developed and constructed for the treatment of homogeneous and particulate materials.

The studies carried out and the measures and solutions adopted up to now have not led to any acceptable results.

### BRIEF SUMMARY OF THE INVENTION

In order to overcome the drawbacks described above, a new device for the sterilization of hospital waste has been developed.

The object of the new hospital waste sterilization device is to ensure the complete sterilization of the waste, so that it can be successively disposed of as solid urban waste.

Another object of the new hospital waste sterilization device is to guarantee the absence of closed containers among the waste to be sterilized.

The new device for the sterilization of hospital waste involves a first trituration, or fine chopping, of the waste introduced into the device, so as to open the waste containers, break up the bigger objects, and break and crush the closed elements. The triturated waste is sprinkled and mixed with high-temperature steam.

Finally, the mass of triturated waste is put into a hermetic container provided with a stirrer, where it is subjected to microwave irradiation and to a high-temperature pressurized steam treatment for a given amount of time.

If, for any reasons, during the process the temperature and/or pressure values fall below minimum preset levels, the entire microwave irradiation and steam flooding process will be started again from the beginning. The combined action of steam and microwaves, together with the continuous stirring, sterilizes completely the whole mass of triturated waste.

The entire process, from the introduction into the triturator until the exit from the stream and microwave treatment container, takes place in an insulated environment, with no contact with the external environment.

The characteristics of the new hospital waste sterilization device will be highlighted in greater detail in the following description of one among many possible applications of the invention, making reference to the enclosed drawings.

### BRIEF DESCRIPTION OF THE SEVERAL VIEWS OF THE DRAWINGS

FIG. 1 is a front view of the device.

FIG. 2 is a top view of the device of FIG. 1.

FIGS. 3 and 4 are side views of the device of FIG. 1.

FIG. 5 shows a cross section of the sterilization chamber of the device of FIG. 1.

### DETAILED DESCRIPTION OF THE INVENTION

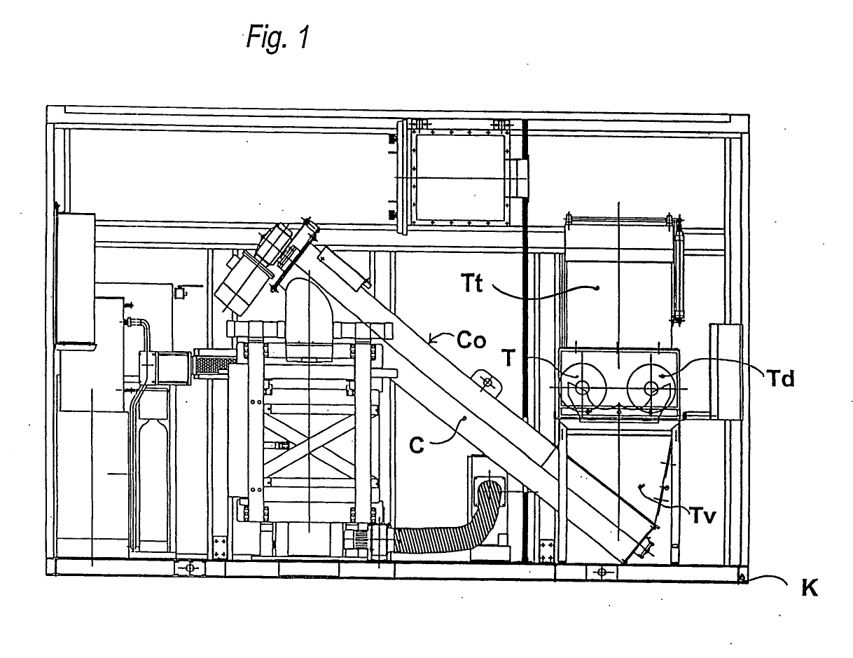

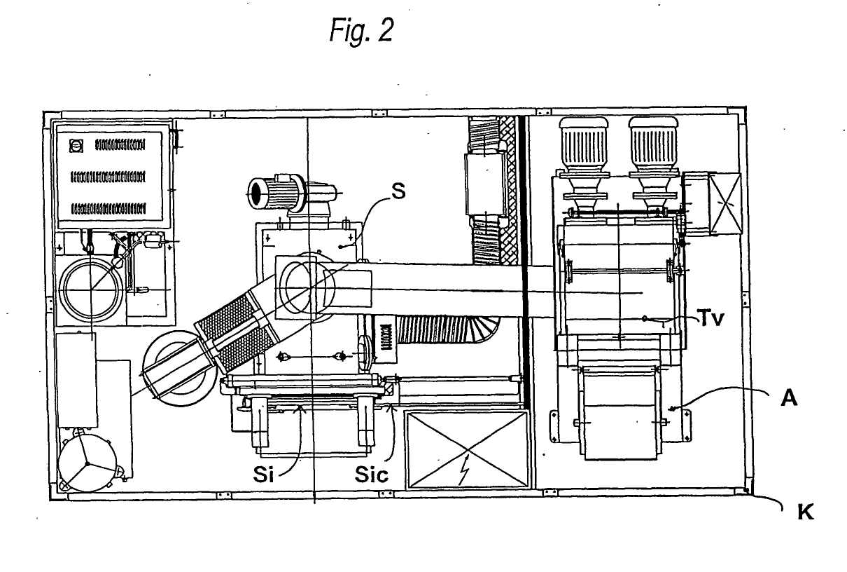

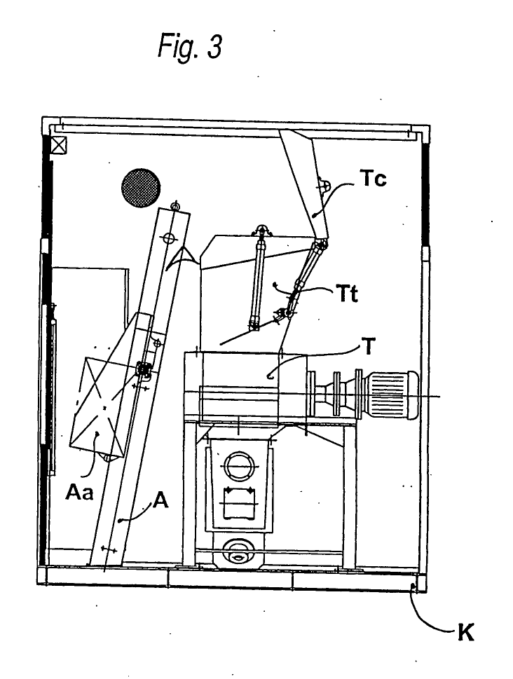

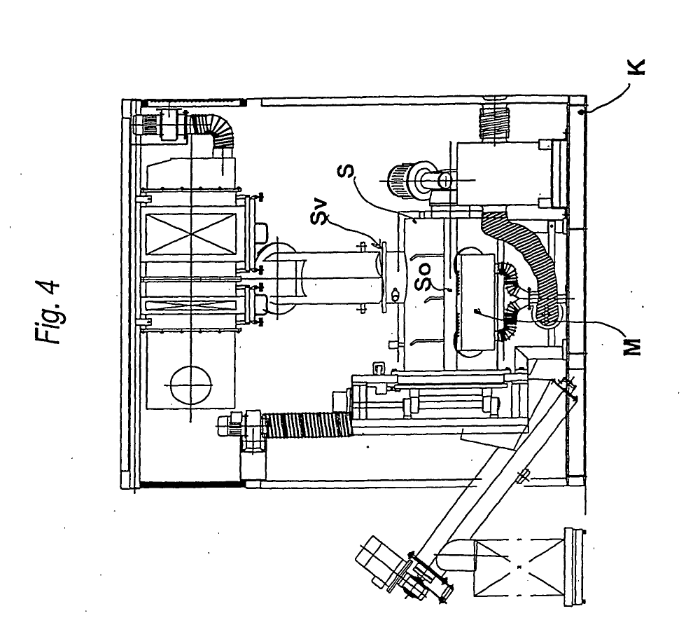

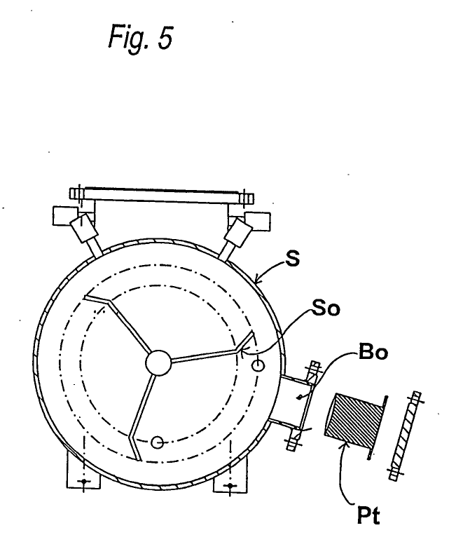

FIGS. 1-5 show schematically the various parts of the new sterilization device, more precisely the loading device (A), the triturator (T) (a device that crushes or grinds in fine particles) with the related hopper (Tt), the auger feeder (C), the sterilization chamber (S), and the microwave generators (M). The loading device (A) is constituted by a basket or container (Aa) that receives the bags or containers of waste to be sterilized and that is then lifted and overturned in the hopper (Tt) of the triturator (T), for example by means of chains or of a fluid-mechanic system.

The triturator (T) comprises a hopper (Tt) with a hermetic cover at the top (Tc) and a series of horizontal toothed drums (Td) positioned side by side at the bottom.

A thrusting device or another device suitable to convey and thrust the waste towards the toothed drums (Td) may be provided inside the hopper (Tt) of the triturator (T), in such a way as to optimize the operation of the triturator (T) and to ensure the correct trituration of the waste.

Said hopper (Tt) is kept under vacuum and the air present inside it is extracted, conveyed towards a so-called “absolute” filter, that is, a filter capable of cutting off 100% by weight of solid particles greater than a stated micron size, and discharged to the external environment.

Under the toothed drums (Td) of the triturator (T) there is a collection compartment (Tv) for the temporary accumulation of triturated waste, provided with optical sensors or other types of sensors for detecting the introduction of waste into the auger feeder (C) and the achievement of the optimal loading level.

The auger feeder (C) is closed inside an inclined duct (Co) and connects the collection compartment (Tv) of the triturator (T) with the upper opening of the sterilization chamber (S). Along the duct (Co) of the auger feeder (C) there are nozzles suitable for introducing steam inside the duct (Co).

The sterilization chamber (S) comprises a preferably cylindrical container provided with two openings, more precisely, an upper opening and another lateral or front opening.

The upper opening of the sterilization chamber (S) is provided with a gate valve (Sv) that separates it hermetically from the auger feeder (C).

Similarly, the lateral or front opening of the sterilization chamber (S) is provided with a mobile gate (Si) that opens and closes completely the mouth of the sterilization chamber (S). In particular, the gate (Si) of the opening of the sterilization chamber (S) is supported by a carriage (Sic) that translates horizontally on guides owing to the action of a preferably pneumatic cylinder. The translation of gate (Si) causes the opening of the sterilization chamber (S) by the action of jacks.

Inside the sterilization chamber (S) there is a stirrer (S) preferably constituted by a horizontal shaft with radial arms or blades.

A rear steam inlet nozzle is positioned on the wall of the sterilization chamber (S), while the microwave generators (M) are arranged on one or more levels, around the sterilization chamber (SThe walls of the sterilization chamber (S), where the microwave generators (M) are positioned, comprise openings plugged by elements (Pt), preferably made of poly-tetra-fluoro- or ethylene or other anti-adhesive materials, in order to achieve the best results from the irradiation process.

Optical, ultrasound or other sensors are positioned in the upper part of the sterilization chamber (S) to detect the maximum loading level of triturated waste in the sterilization chamber (S).

Other temperature and pressure sensors are placed in appropriate positions on the walls of the sterilization chamber (S), in such a way as to have a constant monitoring of temperature and pressure.

All the various electric, electronic and mechanical parts are controlled by an appropriate electronic circuit (not shown in the figures) that supervises the operation of the present device for the sterilization of hospital waste. All the steps of the process, from the introduction of the waste into the hopper (Tt) of the triturator (T) to the exit of the same from the sterilization chamber (S), take place within a closed circuit, with no contact, even aerial, between the waste and the external environment.

To activate the process, the operator deposits one or more closed bags or containers with the waste to be sterilized in the basket or container (Aa) of the loading device (A) and starts the sterilization cycle.

The loading device (A) then lifts the basket or container (Aa) and overturns it into the hopper (Tt) of the triturator, introducing the closed bags or containers into the triturator (T) itself. The sensors present in the upper part of the hopper (Tt) detect the fall of the waste and activate the closing of the hermetic cover (Tc) of the hopper (Tt).

Immediately afterwards, the toothed drums (Td) of the triturator (T) start rotating, thus breaking up the waste and opening and breaking any hollow objects such as bottles and syringes. At the same time the thrusting device, if present, compresses and/or conveys the waste towards said toothed drums (Td), with a motion that will optimize the trituration process.

The waste triturated by the toothed drums (Td) is first accumulated in the collection compartment (Tv) provided under the toothed drums (Td) and then carried by the auger feeder (C).

The auger feeder (C) then conveys the triturated waste towards the mouth of the sterilization chamber (S). While moving along the auger feeder (C) and the connected duct (Co), the waste is sprinkled with steam through the nozzles present on the duct (Co) itself. By this process the auger feeder (C), besides transporting the waste towards the sterilization chamber (S), causes the waste to mix with the steam and allows the steam to penetrate among all the triturated parts of the waste.

At the end of the auger feeder (C) the waste, triturated and pretreated with steam, is introduced into the sterilization chamber (S) through the gate valve (Sv) provided on the upper opening of the sterilization chamber (S) itself.

As the waste is introduced into the sterilization chamber (S), the stirrer (So) present inside the sterilization chamber (S) distributes said waste homogeneously.

The gate valve (Sv) is closed as soon as the maximum loading level of the triturated waste is reached and at this point the actual sterilization phase inside the sterilization chamber (S) is started: the microwave generators (M) are set in operation and high temperature pressurized steam is introduced into the sterilization chamber (S).

During the irradiation of the triturated waste with microwaves and the steam flooding process inside the sterilization chamber (S), the stirrer (So) keeps rotating, thus mixing the triturated waste in such a way as to obtain the maximum microwave irradiation and steam penetration into the triturated waste.

The actions of the microwaves and of the steam make it possible to increase the temperature and pressure values inside the sterilization chamber (S). The action of the stirrer (So) allows the microwaves and the steam to reach all the particles of the triturated waste, thus ensuring that each single part of the waste mass reaches the same pressure and temperature levels reached by the rest of the triturated waste mass.

At the end of the sterilization phase, that is, when the triturated waste has been subjected to sufficient temperatures and pressures for a given amount of time, the microwave generators (M) are stopped, the sterilization chamber (S) is depressurized, and the gate (Si) of the sterilization chamber (S) is opened, in order to allow the discharge of the completely sterilized waste.

This triturated and sterilized hospital waste then undergoes the successive phase of the process, in which it is put into bags in order to be delivered to the dump in the same manner as solid urban waste.

If the temperature and/or the pressure inside the sterilization chamber fall below the minimum levels set, the electronic control circuit of the present device will restart the entire sterilization cycle inside the sterilization chamber (S) again from the beginning, until the required temperature and pressure values are maintained for the necessary amounts of time.

The present hospital waste sterilization device offers considerable advantages and interesting characteristics:
after the introduction of the closed waste bags inside the new device, there is no further contact between the waste and the operators, or between the waste and the external environment;
the containers to be sterilized, like phials, syringes and bottles, are opened and broken up, so that the complete sterilization of all the waste is ensured;
the stirrer inside the sterilization chamber mixes the waste fragments, so that they all reach the same temperature and are thus completely sterilized;
a complete sterilization is ensured by the electronic control circuit, which guarantees that the waste is subjected to a temperature exceeding the minimum temperature set;
the sterilized waste produced by the new device can be treated as solid urban waste and can be delivered to normal dumps;
the cost, the time required by the process, and the risks to the operating personnel are much lower than those in the prior art;
the device described above can be easily fit into standard-size containers and therefore is easy to transport and to install.

Therefore, with reference to the above description and the enclosed drawings, the following claims are put forth.

1. A device for the sterilization of hospital waste, comprising:
a triturator (T) to finely chop the waste, the triturator comprising a hermetic cover (Tc), a hopper (Tt) and toothed drums (Td);
an auger feeder (C) to convey the triturated waste, the auger feeder (C) being provided with nozzles capable of injecting steam into the triturated waste;
a high temperature pressurized steam generator, to provide steam to the nozzles; and
an airtight sterilization chamber (S) equipped with microwave generators and an inner stirrer (So),
wherein the triturator breaks up the waste, and opens and breaks any containers that may be present in the waste, and
wherein the auger feeder (C) conveys the triturated waste from the triturator (T) to the sterilization chamber (S), mixing it with the steam provided by the nozzles, and
wherein the waste contained in the sterilization chamber undergoes microwave irradiation and is mixed with high-temperature steam, the waste being subjected to the combined action of pressure, temperature and microwaves continuously for a suitable amount of time.

2. The device for the sterilization of hospital waste according to claim 1, wherein during the entire process the waste is completely insulated from the external environment, and wherein said hopper (Tt) is kept under vacuum, and wherein the air inside said hopper is extracted, conveyed towards an absolute filter, and discharged into the external environment.

3. The device for the sterilization of hospital waste according to claim 1, wherein the sterilization chamber (S) is provided with a gate valve (Sv) in the upper portion and with a gate (Si), and wherein said sterilization chamber (S) contains a stirring device (So) for mixing the contents of the chamber.

4. The device for the sterilization of hospital waste according to claim 3, wherein said stirring device (So) comprises a shaft that rotates transversally to the chamber and that is equipped with mixing blades.

5. The device for the sterilization of hospital waste according to claim 1, further comprising microwave generators (M) positioned around the sterilization chamber on one or more levels.

6. The device for the sterilization of hospital waste according to claim 5, wherein the walls of the sterilization chamber (S) comprise openings (Bo) in the areas surrounding said microwave generators (M), said openings (Bo) being plugged with elements (Pt) made of an anti-adhesive material.

7. The device for the sterilization of hospital waste according to claim 1, further comprising loading sensors in the hopper (Tt) and in the sterilization chamber (S), and with temperature and pressure sensors in the sterilization chamber (S).

8. The device for the sterilization of hospital waste according to claim 1, wherein all electric, electronic, and mechanical parts of said sterilization device are controlled by an electronic circuit, and wherein the electronic circuit restarts the treatment cycle inside the sterilization chamber if temperature and pressure inside the sterilization chamber fall below predetermined levels.

9. The device for the sterilization of hospital waste according to claim 1, further comprising two waste collection compartments, one under the triturator and the other over the sterilization chamber, the waste being held in the waste collection compartments during the sterilization process inside the sterilization chamber.

10. The device for the sterilization of hospital waste according to claim 1, further comprising a thrusting device positioned inside the hopper (Tt), the thrusting device being suitable for compressing the waste to be sterilized and conveying it towards the toothed drums.

11. The device for the sterilization of hospital waste according to claim 1, wherein the device can be fit into a modular container, in order to be easily transported and installed.
